(19) United States
(12) Patent Application Publication
(10) Pub. No.: US 2002/0006420 A1
(43) Pub. Date: Jan. 17, 2002

(54) USE OF A 2-AMINO-ALKANE POLYOL AS AGENT FOR TREATING SKIN AGEING SIGNS

(76) Inventors: MICHEL PHILIPPE, WISSOUS (FR); BRUNO BERNARD, NEUILLY-SUR-SEINE (FR); QUINTINO GAETANI, CLICHY-SOUS-BOIS (FR)

Correspondence Address: OBLON SPIVAK MCCLELLAND MAIER & NEUSTADT, 1755 JEFFERSON DAVIS HIGHWAY, FOURTH FLOOR, ARLINGTON, VA, 22202, US

(21) Appl. No.: 09/485,191

(22) PCT Filed: Aug. 6, 1998
(86) PCT No.: PCT/FR98/01755

(30) Foreign Application Priority Data
Aug. 7, 1997 (FR) 97 10142

(51) Int. Cl. A61K 7/135; A61K 6/00; A61F 13/00; A61K 9/70; A61K 7/00; A61K 7/021

(52) U.S. Cl. 424/401; 424/443; 514/844; 424/62; 424/63

(57) ABSTRACT

The invention concerns the use, as main active principle in a composition or for preparing a pharmaceutical composition, of an effective amount of at least a 2-amino-alkane polyol, or one of its derivatives for treating skin ageing signs.

The present invention relates to the use, as active ingredient in a cosmetic composition or for the preparation of a pharmaceutical composition, of an effective quantity of at least one 2-amino-1,3-alkanediol, or of one of its derivatives, intended for treating the signs of skin ageing as well as a nontherapeutic method of treating skin ageing.

Skin ageing, resulting from effects of intrinsic or extrinsic factors on the skin, is manifested by the appearance of wrinkles and fine lines, by the yellowing of the skin which develops a wrinkled appearance accompanied by the appearance of pigmented spots, by the disorganization of the elastin and collagen fibres causing a loss of elasticity, of suppleness and of firmness and by the appearance of telangiectasia.

Some of these signs of ageing are more particularly linked to intrinsic or physiological ageing, that is to say to the “normal” ageing linked to age, whereas others are more specific to extrinsic ageing, that is to say to ageing caused in general by the environment; this includes more particularly photoageing due to exposure to the sun, to light or to any other radiation.

The invention relates to intrinsic or physiological ageing as well as to extrinsic ageing.

The changes in the skin resulting from intrinsic or physiological ageing are the consequence of a genetically programmed senescence where endogenous factors come into play. This intrinsic ageing causes in particular a slowing down of the renewal of the skin cells. Histologically, the skin is made thin overall, both at the epidermal and the dermal level. The density of the fibrous macromolecules of the dermis (elastin and collagen) is reduced.

By contrast, extrinsic ageing causes adverse clinical changes such as thick wrinkles and the formation of a flabby and/or weather-beaten skin, and histopathological changes such as an excessive accumulation of elastic material in the upper dermis and degeneration of the collagen fibres.

Substances which make it possible to eliminate or reduce the effect of skin ageing, and in particular to prevent the slowing down of the renewal of the skin cells have been sought for many years in the cosmetic or pharmaceutical industry.

Various agents intended for combating skin ageing are known in the prior art.

Thus, U.S. Pat. No. 4,603,146 describes the use of retinoic acid and of its derivatives in cosmetic compositions, for combating skin ageing.

Moreover, many patents and publications (see for example application EP-A-413,528) as well as many commercially available cosmetic compositions teach the use of α-hydroxy acids such as lactic acid, glycolic acid or citric acid for treating skin ageing.

Finally, the beta-hydroxy acids and more especially salicylic acid as well as its derivatives are known for their desquamatory properties (see the documents WO-A-93/10756 and U.S. Pat. No. 4,767,750).

All these compounds have an action against skin ageing, consisting of a desquamation, that is at least to say the elimination of the “dead” cells situated at the surface of the stratum corneum. This desquamatory property is also called, often wrongly, keratolytic property. However, these compounds also exhibit side effects which consist of pricking, tightness, overheating and redness which are unpleasant for the user.

It can therefore be seen that the need remains for antiageing agents whose action is at least as effective as that of the prior art compounds, but which do not exhibit their disadvantages.

Surprisingly, the Applicant has now discovered that certain compounds of the 2-aminoalkane polyol type have an effect on skin ageing.

The subject of the invention is therefore the use of a 2-aminoalkane polyol of formula (I) (below), or of one of its derivatives, in a cosmetic or dermatological composition for topical application, intended for treating certain signs of endogenous and/or exogenous ageing.

This treatment may be carried out preventively and/or curatively.

The compounds of the 2-aminoalkane polyol type or their derivatives may form part of these compounds of the tissues which are called by the very broad term of sphingolipids.

Among these sphingolipids, the N-acylated derivatives based on sphinganine [(2S,3R)-2-amino-1,3-octadecanediol] are ceramides which are predominantly present in the hair, whereas analogous derivatives based on phytosphingosine [(2S,3S,4R)-2-amino-1,3,4-octadecanetriol], other ceramides, form part of the lipids which are predominantly present in the stratum corneum of the skin.

Ceramides are used in cosmetics in the natural or synthetic form in compositions intended, for example, for strengthening the barrier effect of the stratum corneum in order to reduce the water loss and therefore the drying of the skin (GB 2,178,312, GB 2,213,723, EP 227,994, EP 282,616, EP 556,957).

They are also used in cosmetic compositions for their properties which confer better elasticity on the skin (EP 500,437) or in compositions for hair use for strengthening the hair and/or for repairing the damage caused by the continuous attacks to which it is subjected.

To the knowledge of the Applicant, there has never been described or even suggested for the 2-aminoalkane polyols of formula (I), or their derivatives, an effect on the proliferation of the cells, let alone of the keratinocytes.

It is on the basis of this new property of these amino polyols that the Applicant has been able to show that these compounds also have an effect on skin ageing.

The invention relates to the use, in a cosmetic composition or for the preparation of a pharmaceutical composition intended for treating the signs of skin ageing, of at least one compound of general formula (I):

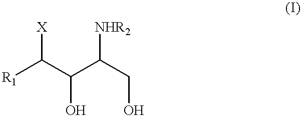

in which R₁ represents a saturated or unsaturated, optionally hydroxylated, linear or branched hydrocarbon radical having from 4 to 28 carbon atoms, R₂ represents a hydrogen atom or the radical

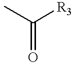

in which R₃ represents a saturated or unsaturated, linear or branched hydrocarbon radical having from 1 to 11 carbon atoms or a saturated or unsaturated, hydroxylated, linear or branched hydrocarbon radical having from 1 to 29 carbon atoms, it being possible for the hydroxyl to be esterified by a saturated or unsaturated, linear or branched acyl chain having from 2 to 30 carbon atoms;

X represents a hydrogen atom or the OH radical;

or of at least one of its optical isomers or one of the diastereoisomers.

R₁ preferably contains from 6 to 22 carbon atoms and still more preferably from 10 to 18 carbon atoms.

Preferably, R₂ represents H or a radical

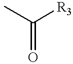

and R₃ represents a saturated or unsaturated, linear or branched hydrocarbon radical having from 1 to 9 carbon atoms or a saturated or unsaturated, hydroxylated, linear or branched hydrocarbon radical having from 1 to 21 carbon atoms.

Preferably, R₁ represents a saturated and/or linear hydrocarbon radical, more particularly a saturated linear hydrocarbon radical.

Preferably, R₁ represents a saturated hydrocarbon radical having 14 carbon atoms, and still more preferably R₁ represents a saturated linear hydrocarbon radical having 14 carbon atoms.

The agent promoting the treatment and/or the prevention of ageing may be a mixture of compounds of formula (I), for which R₁ and/or R₂ are chains of different lengths.

These compounds may be used in the form of a pure isomer or of a mixture of isomers.

These compounds have the advantage of not inducing disturbing side effects, which renders their use risk-free for the user.

There may be mentioned as compounds of formula (I), which can be used according to the invention:

2-amino-1,3-octadecanediol,

2-N-acetylamino-1,3-octadecanediol,

2-N-octanoylamino-1,3-octadecanediol,

2-N-(2-hydroxyhexadecanoyl)amino-1,3-octadecanediol,

2-N-(2-hydroxydocosanoyl)amino-1,3-octadecanediol,

2-amino-1,3,4-octadecanetriol,

2-N-(2-hydroxyhexadecanoyl)amino-1,3,4-octadecanetriol,

2-N-hexanoylamino-1,3-octadecanediol,

2-N-octanoylamino-1,3,4-octadecanetriol.

Preferably, 2-N-acetylamino-1,3-octadecanediol or 2-N-octanoylamino-1,3-octadecanediol is used according to the invention.

The subject of the invention is therefore the use of a 2-amino-1,3-alkanediol of formula (I) (below), or of one of its derivatives, in a cosmetic or dermatological composition for topical application, intended for treating wrinkles and/or fine lines and/or yellowing of the skin and/or the wrinkled appearance of the skin and/or the pigmented spots on the skin and/or telangiectasia and/or the loss of elasticity, suppleness and/or firmness of the skin.

The quantity of compounds of formula (I) which can be used according to the invention of course depends on the nature of the compound used, its physicochemical properties and its mode of application. Persons skilled in the art know how to adjust the quantity of compound of formula (I) to be used depending on the needs.

As a guide, the compounds of formula (I) may be used in concentrations by weight of between 10⁻⁴% and 20%, and preferably between 10⁻³% and 10% in the composition.

When the compound of formula (I) is used in a composition which has to be applied to the skin, this composition may be provided in all the galenic forms which are normally used.

For a topical application to the skin, the composition may be in the form in particular of an aqueous, alcoholic, aqueous-alcoholic or oily solution or a dispersion of the lotion or serum type, an emulsion having a liquid or semiliquid consistency of the milk type which is obtained by dispersing a fatty phase in an aqueous phase (O/W) or conversely (W/O), or a suspension or an emulsion having a soft consistency of the cream, foam or aqueous or anhydrous gel type, or microcapsules or microparticles, or a vesicular dispersion of the ionic and/or nonionic type. It may also take the form of an aerosol composition also comprising a propellant under pressure.

Whatever its form, this composition is prepared according to the customary methods.

The quantities of the various constituents of the composition according to the invention are those conventionally used in the fields considered.

When the composition is an emulsion, the proportion of the fatty phase may range from 5% to 80% by weight, and preferably from 5% to 50% by weight relative to the total weight of the composition. The oils, waxes, emulsifiers and coemulsifiers used in the composition in the form of an emulsion are chosen from those conventionally used in the cosmetic field. The emulsifier and coemulsifier are present in the composition in a proportion ranging from 0.3% to 30% by weight, and preferably from 0.5 to 20% by weight relative to the total weight of the composition. The emulsion may, in addition, contain lipid vesicles.

When the composition is a solution or an oily gel, the fatty phase may represent more than 90% of the total weight of the composition.

It is also possible to envisage the composition comprising at least one compound of formula (I) as active ingredient intended for treating and/or preventing skin ageing being in liposomal form, as described in particular in Patent application WO 94/22468 filed on Oct. 13, 1994 by the company Anti Cancer Inc.

This composition may also contain customary adjuvants in the cosmetic field, such as hydrophilic or lipophilic gelling agents, hydrophilic or lipophilic additives, preservatives, antioxidants, solvents, perfumes, fillers, screening agents, odour absorbers and colouring matter. The quantities of these different adjuvants are those conventionally used in the cosmetic field, and for example from 0.01% to 10% of the total weight of the composition. These adjuvants, depending on their nature, may be introduced into the fatty phase, into the aqueous phase and/or into the lipid spherules.

As oils or waxes which can be used in the invention, there may be mentioned inorganic oils (liquid paraffin), vegetable oils (liquid fraction of shea butter, sunflower oil), animal oils (perhydrosqualene), synthetic oils (purcellin oil), silicone oils or waxes (cyclomethicone) and fluorinated oils (perfluoropolyethers), beeswax, carnauba wax or paraffin wax. It is also possible to add fatty alcohols and fatty acids (stearic acid) to these oils.

As emulsifiers which can be used in the invention, there may be mentioned, for example, glycerol stearate, polysorbate 60 and the PEG-6/PEG-32/glycol stearate mixture sold under the name Tefose®63 by the company Gattefosse.

As solvents which can be used in the invention, there may be mentioned the lower alcohols, in particular ethanol and isopropanol, propylene glycol.

As hydrophilic gelling agents which can be used in the invention, there may be mentioned the carboxyvinyl polymers (carbomer), the acrylic copolymers such as copolymers of acrylates/alkyl-acrylates, polyacrylamides, polysaccharides such as hydroxypropylcellulose, natural gums and clays, and, as lipophilic gelling agents, there may be mentioned modified clays such as bentones, metal salts of fatty acids such as aluminium stearates and hydrophobic silica, ethyl cellulose, polyethylene.

The composition may contain other hydrophilic active agents such as proteins or protein hydrolysates, amino acids, polyols, urea, allantoin, sugars and sugar derivatives, water-soluble vitamins, plant extracts and hydroxy acids.

As lipophilic active agents, there may be used retinol (vitamin A) and its derivatives, tocopherol (vitamin E) and its derivatives, essential fatty acids, ceramides other than the compounds of formula (I), essential oils, salicylic acid and its derivatives.

In particular, it is also possible to use, in combination with the compound of formula (I) used according to the invention, compounds which further enhance the activity on the treatment and/or prevention of ageing, and which have already been described for this activity.

Among the latter compounds, there may be mentioned more particularly with no limitation being implied:

keratolytic agents such as alpha- and beta-hydroxycarboxylic or beta-ketocarboxylic acids, their salts, amides or esters and more particularly the hydroxy acids such as glycolic acid, lactic acid, salicylic acid, citric acid and in general the fruit acids, and 5-(n-octanoyl)salicylic acid;

anti-free-radical agents such as alpha-tocopherol or its esters, superoxide dismutases, certain metal chelators or ascorbic acid and its esters;

anti-acne agents such as retinoic acid or benzoyl peroxide.

It is moreover possible to combine with the compounds of formula (I) antagonists of substance P and/or of CGRP (Calcitonin Gene Related Peptide) such as Iris pallida and the salts of strontium, in particular the chlorides and nitrates of strontium, or antagonists of substance P and/or of CGRP such as those described in French Patent Applications FR-2,719,474 and FR-2,729,855. Such a combination makes it possible to ensure perfect tolerance of these compositions, even by very sensitive skins.

It is possible to use at least one of the compounds corresponding to the formula (I) in the preparation of a pharmaceutical, particularly dermatological, composition intended for treating and/or preventing skin ageing.

In general, these pharmaceutical compositions are in particular distinguishable from the cosmetic compositions by the quantity of active agent which they contain. Persons skilled in the art, in this case, know how to determine the quantity of active agent which can be used depending on the result sought but also on the mode of administration envisaged.

As a guide, the compound of formula (I) may be used in the preparation of a pharmaceutical composition at a concentration of between 0.01% and 20% and preferably between 0.1% and 10% by weight relative to the weight of the composition.

The cosmetic or pharmaceutical composition according to the invention may be applied to the skin, generally the skin of the face and/or of the neck and/or of the hands, and optionally left in contact for several hours and optionally rinsed. It is possible, for example, to apply the composition containing an effective quantity of at least one compound of formula (I) in the evening and to keep it in contact for the whole night and optionally for washing to be carried out in the morning. These applications may be repeated daily for one or more months depending on the individual.

Thus, the subject of the present invention is also a method for cosmetic treatment of the human skin, characterized in that it consists in applying to at least part of the skin of the human body a cosmetic composition comprising an effective quantity of at least one compound of formula (I), in leaving it in contact with the skin and in optionally rinsing.

The following examples and compositions illustrate the invention without limiting it in any way. In the compositions, the proportions indicated are percentages by weight unless otherwise indicated.

### EXAMPLE 1

### Measurement of the Proliferative Effect of 2-amino-1,3-octadecanediol and its Derivatives on Keratinocytes in Culture

HaCat cells (Boukamp et al., J. Cell Biol. Vol. 106, March 1988, 761-771) are cultured in DMEM medium (company Gibco) containing amino acids (mixture of nonessential amino acids) at the concentration of 0.1 mM, penicillin and streptomycin at the respective concentrations of 50 International Units per milliliter and 50 μg/ml, glutamine at the concentration of 2 mM, sodium pyruvate at the concentration of 1 mM and foetal calf serum at 10%. These cells were inoculated at the density of 10,000 cells per well into the 24 wells of multiwell-type plates (Costar, France).

24 hours after inoculation, the cells are brought into contact with various compounds tested in a KBM medium (company Clonetics) containing 0.15 mM of calcium ion, insulin at 5 μg/ml, hydrocortisone at 0.5 μg/ml and lipid-free bovine serum albumin at 1 μg/ml.

Cell counting is carried out 5 days after the addition of the various ceramides using a cell counter of the Coulter Counter type.

The various compounds are tested at 0.5 nM, 5 nM, 50 nM and 500 nM.

The results, expressed as a percentage, represent the increase in the number of cells relative to the control, that is to say relative to a culture carried out under the same conditions in the absence of sphinganine.

The various compounds tested are:

A: 2-amino-1,3-octadecanediol,

B: 2-N-(2-hydroxyhexadecanoyl)amino-1,3-octadecanediol,

C: 2-N-acetylamino-1,3-octadecanediol,

D: 2-N-octanoylamino-1,3-octadecanediol.

| | | A | B | C | D |
|---|---|---|---|---|---|
| 0.5 | nM | 7 | 25 | 17 | 15 |
| 5 | nM | 15 | 36 | 34 | 18 |
| 50 | nM | 17 | 42 | 29 | 31 |
| 500 | nM | 30 | 22 | 35 | 27 |

These results show that 2-amino-1,3-octadecanediol and its derivatives have a proliferative activity on keratinocytes in culture.

### EXAMPLE 2

### Measurement of the Effect of 2-amino-1,3-octadecanediol and its Derivatives on Cell Viability

HaCat cells are cultured as above.

They are then inoculated at the density of 20,000 cells per well into the 24 wells of multiwell-type plates (Costar, France).

24 hours after inoculation, the cells are brought into contact with various compounds tested in a KBM medium (company Clonetics) containing insulin at 5 μg/ml and hydrocortisone at 0.5 μg/ml.

The cell viability is evaluated 48 hours later by the measurement of mitochondrial respiration. This is carried out with the aid of an XTT kit sold by the company Boehringer and according to the supplier's instructions.

Compounds A and B of the preceding example were tested at the concentration of 50 nM, as well as a derivative based on sphingenine: N-palmitoylsphingenine from Sigma (C16H).

The results, expressed as a percentage, represent the increase in the number of cells relative to the control, that is to say relative to a culture carried out under the same conditions in the absence of alkanediol compounds.

| Control | 0 |
|---|---|
| A | +17 |
| B | +20 |
| C16H | −25 |

The results show that 2-amino-1,3-octadecanediol (A) and 2-N-(2-hydroxyhexadecanoyl)amino-1,3-octadecanediol (B) increase the viability of the keratinocytes in culture, whereas N-palmitoyl-sphingenine (C16H) has a cytotoxic effect on the cells in culture.

### EXAMPLE 3

### Measurement of the Effect of 2-amino-1,3-octadecanediol and its Derivatives on the Cell Cycle

HaCat cells are cultured as above.

They are then inoculated at the density of 20,000 cells per well into the 24 wells of multiwell-type plates (Costar, France).

24 hours after inoculation, the cells are brought into contact with various compounds tested in a KBM medium (company Clonetics) containing insulin at 5 μg/ml and hydrocortisone at 0.5 μg/ml, and 5-bromo-2′-deoxyuridine (BrdU) at 1 μM.

The phase of the cell cycle is evaluated 48 hours later by the measurement of the incorporation of BrdU into the genomic deoxyribonucleic acid. This is carried out with the aid of a “BrdU fast” kit sold by the company Boehringer and according to the supplier's instructions.

Compounds A and B of the preceding example were tested at the concentration of 50 nM.

The results, expressed as a percentage, represent the increase in the number of cells which entered the S phase relative to the control, that is to say relative to a culture carried out under the same conditions in the absence of alkanediol compounds.

| | 72 hours | 96 hours |
|---|---|---|
| Control | 0 | 0 |
| A | 36 | 41 |
| B | 17 | 43 |

The results show that 2-amino-1,3-octadecanediol (A) and 2-N-(2-hydroxyhexadecanoyl)amino-1,3-octadecanediol (B) increase the number of keratinocytes which entered the S phase, thus indicating stimulation of the cellular mitotic activity.

### EXAMPLE 4

### Effect of 2-amino-1,3-octadecanediol and its Derivatives on Cell Viability in the Presence of a Compound Based on Sphingenine N-acetylsphingenine

HaCat cells are cultured as in Example 2.

24 hours after inoculation, the cells are brought into contact with various compounds tested in a KBM medium (company Clonetics) containing insulin at 5 μg/ml and hydrocortisone at 0.5 μg/ml.

The cells are cultured in the presence of the 2-amino-1,3-octadecanediol to be tested until the cell viability is evaluated.

The N-acteylsphingenine at the concentration of 10 μM is added to the culture medium over 2 hours at the beginning of the experiment.

The cell viability is evaluated 48 hours later by the measurement of mitochondrial respiration. This is carried out with the aid of an XTT kit sold by the company Boehringer and according to the supplier's instructions.

The experiment was carried out with the following compounds at the concentration of 50 nM:

C: 2-amino-1,3-octadecanediol,

D: 2-N-(2-hydroxyhexadecanoyl)amino-1,3-octadecanediol,

in comparison with the culture medium without alkanediol and without N-acetylsphingenine (A), and with a medium containing only N-acetylsphingenine (B).

The results, expressed as a percentage, represent the number of cells relative to the control (A), that is to say relative to a culture carried out under the same conditions in the absence of alkanediol compounds and of N-acetysphingenine.

| | |
|---|---|
| A | 100 |
| B | 57 |
| C | 72 |
| D | 78 |

N-Acetylsphingenine causes a decrease in cell viability. The presence, in the culture medium, of a 2-amino-1,3-octadecanediol tends to counter this effect.

### EXAMPLE 5

Examples of compositions to be applied to the skin. These compositions may be prepared by simple mixing. The percentages are given by weight of active material relative to the total weight of the composition.

| | |
|---|---|
| Moisturizing oil-in-water emulsion: | |
| Maize germ oil | 2% |
| Glycerol monostearate | 3% |
| Polyethylene glycol 400 | 3% |
| Carbopol 491 ® marketed by the | 0.2% |
| company GOODRICH | |
| Isopropyl myristate | 3% |
| N-octanoylamino-l,3-octadecanediol | 0.1% |
| Cetyl alcohol | 3% |
| Stearyl alcohol | 3% |
| NaOH | 0.008% |
| Propylene glycol | 5% |
| Preservatives | qs |
| Water | qs 100 |
| Moisturizing water-in-oil emulsion: | |
| Paraffin oil | 10% |
| Protegin X marketed by the | 20% |
| company Goldschmidt | |
| Sunflower oil | 15% |
| Flavouring composition | 1% |
| N-acetylamino-l,3-octadecanediol | 0.05% |
| Magnesium sulphate | 0.5% |
| Glycerol | 5% |
| Cetrol HE marketed by the | 4% |
| company Henkel | |
| Preservative | qs |
| Demineralized water | qs 100 |
| Aqueous gel | |
| Carbopol 940 ® marketed by the | 0.6% |
| company GOODRICH | |
| Transcutol ® marketed by the | 5% |
| company GATTEFOSSE | |
| Triethanolamine | 0.3% |
| Propylene glycol | 3% |
| NaOH | 0.007% |
| N-octanoylamino-l,3-octadecanediol | 0.1% |
| Preservative | qs |
| Water | qs 100 |
| Nonionic liposome-containing cream | |
| Carbopol 940 ® marketed by the | 0.2% |
| company GOODRICH | |
| Transcutol ® marketed by the | 3% |
| company GATTEFOSSE | |
| Triethanolamine | 0.2% |
| Polyglyceryl-3-cetyl ether | 3.8% |
| β-sitosterol | 3.8% |
| Dicetyl phosphate | 0.4% |
| NaOH | 0.007% |
| N-acetylamino-1,3-octadecanediol | 0.15% |
| Sunflower oil | 35% |
| Perfume | qs |
| Preservative | qs |
| Water | qs 100 |

1. Use, in a cosmetic composition or for the preparation of a pharmaceutical composition intended for treating the signs of skin ageing, of an effective quantity of at least one compound of general 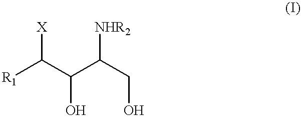
formula (I):
in which R₁ represents a saturated or unsaturated, optionally hydroxylated, linear or branched hydrocarbon radical having from 4 to 28 carbon atoms;
R₂ represents a hydrogen atom or the radical 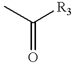
in which R₃ represents a saturated or unsaturated, linear or branched hydrocarbon radical having from 1 to 11 carbon atoms or a saturated or unsaturated, hydroxylated, linear or branched hydrocarbon radical having from 1 to 29 carbon atoms, it being possible for the hydroxyl to be esterified by a saturated or unsaturated, linear or branched acyl chain having from 2 to 30 carbon atoms;
X represents a hydrogen atom or the OH radical;
or of at least one of its optical isomers or one of the diastereoisomers.

2. Use according to claim 1, characterized in that R₁ represents a hydrocarbon radical having from 6 to 22 carbon atoms.

3. Use according to either of claims 1 and 2, characterized in that R₁ represents a saturated hydrocarbon radical.

4. Use according to any one of the preceding claims, characterized in that R₁ represents a hydrocarbon radical having from 10 to 18 carbon atoms.

5. Use according to any one of claims 1 to 4, characterized in that R₁ represents a linear hydrocarbon radical.

6. Use according to any one of the preceding claims, characterized in that R₂ represents H.

7. Use according to any one of claims 1 to 5, characterized in that R₂ represents a radical 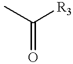
and R₃ represents a saturated or unsaturated, linear or branched hydrocarbon radical having from 1 to 9 carbon atoms or a saturated or unsaturated, hydroxylated, linear or branched hydrocarbon radical having from 1 to 21 carbon atoms.

8. Use according to any one of the preceding claims, characterized in that the quantity of compound of formula (I) is between 10⁻⁴% and 20% by weight relative to the weight of the composition.

9. Use according to the preceding claim, characterized in that the quantity of compound of formula (I) is between 10⁻³% and 10% by weight relative to the weight of the composition.

10. Use according to any one of the preceding claims, characterized in that the compound of formula (I) is chosen from:
2-amino-1,3-octadecanediol,
2-N-acetylamino-1,3-octadecanediol,
2-N-octanoylamino-1,3-octadecanediol,
2-N-(2-hydroxyhexadecanoyl)amino-1,3-octadecanediol.
2-N-(2-hydroxydocosanoyl)amino-1,3-octadecanediol,
2-amino-1,3,4-octadecanetriol,
2-N-(2-hydroxyhexadecanoyl)amino-1,3,4-octadecanetriol,
2-N-hexanoylamino-1,3-octadecanediol,
2-N-octanoylamino-1,3,4-octadecanetriol.

11. Use according to the preceding claim, characterized in that the compound of formula (I) is chosen from 2-N-acetylamino-1,3-octadecanediol and 2-N-octanoylamino-1,3-octadecanediol.

12. Use according to any one of the preceding claims, characterized in that it is intended for treating wrinkles and/or fine lines.

13. Use according to any one of the preceding claims, characterized in that it is intended for treating yellowing of the skin.

14. Use according to any one of the preceding claims, characterized in that it is intended for treating the wrinkled appearance of the skin.

15. Use according to any one of the preceding claims, characterized in that it is intended for treating the pigmented spots on the skin.

16. Use according to any one of the preceding claims, characterized in that it is intended for treating telangiectasia.

17. Use according to any one of the preceding claims, characterized in that it is intended for treating the loss of elasticity, suppleness and/or firmness of the skin.

18. Method for cosmetic treatment of the human skin, characterized in that it consists in applying to at least part of the skin of the human body a cosmetic composition comprising an effective quantity of at least one compound of formula (I) as defined in any one of claims 1 to 11, in leaving it in contact with the skin and in optionally rinsing.
